(19) United States
(12) Patent Application Publication
(10) Pub. No.: US 2019/0038723 A1
(43) Pub. Date: Feb. 7, 2019

(54) Inhibiting UVB-Irradiation Damage By Targeting Nitric Oxide Synthases (cNOS)

(71) Applicant: Ohio University, Athens, OH (US)

(72) Inventors: Shiyong Wu, Athens, OH (US); Lingying Tong, Athens, OH (US)

(73) Assignee: Ohio University, Athens, OH (US)

(21) Appl. No.: 16/073,856

(22) PCT Filed: Feb. 9, 2017
(86) PCT No.: PCT/US2017/017166

§ 371 Date: Jul. 30, 2018

Related U.S. Application Data
(60) Provisional application No. 62/293,860, filed on Feb. 11, 2016.

(51) Int. Cl. A61K 38/44; A61K 31/198; A61K 9/00; A61P 39/00

(52) U.S. Cl. CPC A61K 38/44; C12Y 114/13039; A61P 39/00; A61K 9/0014; A61K 31/198

(57) ABSTRACT

Compositions and methods for inhibition UVB-irradiation damage by targeting constitutive nitric oxide synthases (cNOS), including endothelial NOS (eNOS) and neuronal NOS (nNOS) are described.

### RELATED APPLICATIONS

This application claims priority to U.S. Provisional Application Ser. No. 62/293,860 filed under 35 U.S.C. § 111(b) on Feb. 11, 2016, the disclosure of which is incorporated herein by reference in its entirety.

### STATEMENT REGARDING FEDERALLY SPONSORED RESEARCH

This invention was made with government support under Grant No. CA086928 awarded by the National Institutes of Health. The government has certain rights in the invention.

### BACKGROUND OF THE INVENTION

While extensive studies have been done on UV-activated pathways that regulate DNA-damage repair, cell growth arrest, and induction of apoptosis, the role of autophagy is less well understood.

### SUMMARY OF THE INVENTION

Described herein is the use of cNOS inhibitors as chemopreventive agent for UVB-induced skin cancer formation and progress. Among the benefits are that cNOS inhibitors not only reduced DNA damage, but can also reduce viability of damaged cells. The cNOS inhibitors also reduce apoptosis/necrosis of normal skin tissues after UVB-irradiation.

Another benefit of using cNOS inhibitor as chemopreventive agent is that the cNOS inhibitors reduce skin cancer formation without sensitizing the skin to UVB.

### BRIEF DESCRIPTION OF THE DRAWINGS

The patent or application file may contain one or more drawings executed in color and/or one or more photographs. Copies of this patent or patent application publication with color drawing(s) and/or photograph(s) can be provided by the U.S. Patent and Trademark Office upon request and payment of the necessary fees.

FIG. 1: Autophagy in COS-1 and HEK293T cells after UVB. COS-1 and HEK293T cells were transfected with pcDNA3.1-GFPLC3B plasmid. At 24 h post transfection, the cells were exposed to UVB radiation (50 mJ/cm²). At 6 h post-UVB, the aggregation of LC3 (pointed by arrow) in the cells were observed with fluorescent microscope.

FIG. 2: Model for UVB induced cNOS-NF-κB-IKKα regulated autophagy.

FIG. 3: Quantitative real-time PCR analysis of mRNA expression of IKKα in HaCaT human keratinocyte. The cells were pre-treated with DMSO or actinomycin 0 (Act.D, 5 μg/mL) and treated with of UVB (50 mJ/cm²). The mRNA expression was normalized to GAP OH expression and depicted as fold change vs. DMSO control.

FIG. 4: Scheme of IKKα promoter constructs.

FIG. 5: Model for role of cNOS-mediated DNA damage and autophagy in UVB-induced skin carcinogenesis.

FIGS. 6A-6B: UVB-induced HaCaT cells transformation. The cells were sham or UVB treated every other day for 10 days. The transformation of cells was analyzed by soft-agar assay. FIG. 6A: the arrows point at the transformed cell colonies growing in the soft-agar. FIG. 6B: At day 6, the cells were solubilized in Cytoselect buffer (Cell Biolabs, Inc.) and cell transformation was determined by fluorescence intensity using a fluorescent platereader. *: p value of <0.05 UVB vs. control.

FIG. 7: Polymer-based 3D culture scaffold prepared by electro-spinning technology. Confocal microscopic imaging of the structure of PCL/acetone nanofibers made by electrospinning Scale bar is 10.

FIGS. 8A-8C: Expression of vimentin in 2D & 3D cultured cells. H1299 cells were seeded on a 2D plate (FIG. 8A) or 3D culture scaffold (FIG. 8B). The cells were stained by calcein green and imaged by confocal microscope. The cells were harvested at 1 to 10 days postseeding. The expression of vimentin was determined using western blot analysis (FIG. 8C). Scale bar is 100 tm.

FIG. 9: Illustration of two-layer 3D co-culture system.

FIGS. 10A-10B: UVB-induced DNA damage in HaCaT cells. The image (FIG. 10A) was captured by Cytation 3 Imaging Analyzer (BioTek, Winooski, Vt.); and, the DNA density (FIG. 10B) was analyzed by ImageJ (Version 1.46r. NIH) with Comet Assay Plugin (Pathology & Lab Med UNC-CH). *: p-value<0.05 vs. DMSO alone; **: p-value<0.05 vs. NAME. ***: p-value<0.05 vs. UV+DMSO. The data represents an average of DNA damage analysis for more than five cells.

FIG. 11: UVB-induced HaCaT cells transformation. The cell transformation was determined using a Transformation assay kit (Cell Biolabs, Inc.). *: p-value<0.05 vs. control. **: p-value<0.05 vs. UVB alone. The data represents an average of 3 sets of independent experiments.

### DETAILED DESCRIPTION OF THE INVENTION

It is now shown herein that UVB-induced activation of cNOS enhances the development of skin cancer by both increasing the extent of cell damage and increasing the probability that damaged cells can survive.

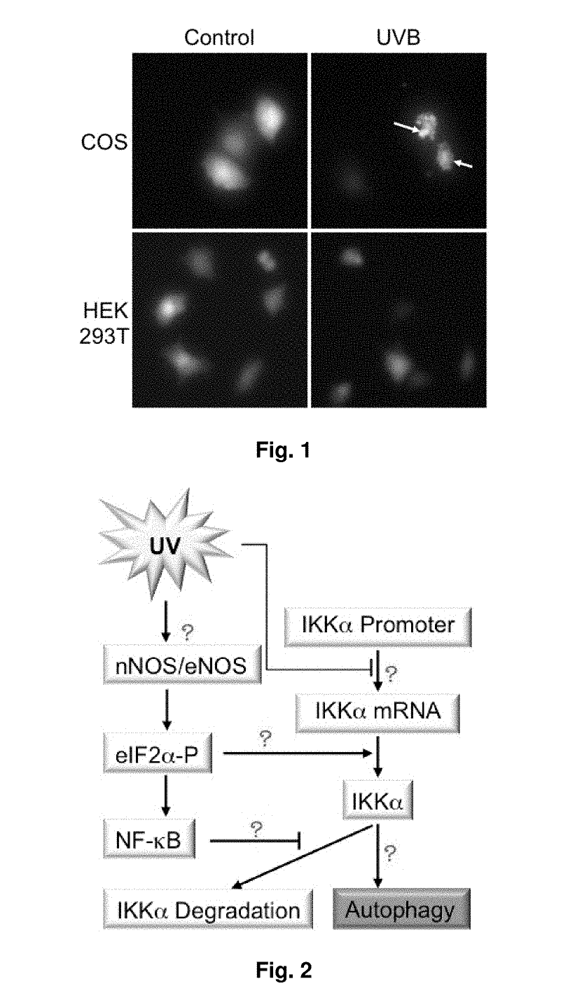

It is now also shown herein that cNOS inhibitors can protect against formation of skin cancers. A desirable property of cNOS as a target is that it only affects UVB-induced (and not TNFα-induced) NF-κB activation. UVB irradiation also increases autophagy in a cNOS-dependent manner (FIG. 1). UVB-induced NF-κB activation is dependent on cNOS activation. Thus, cNOS is a good target for chemoprevention and treatment of UVB-induced cancer formation and progress.

Lentivirus system (Addgene) can be used for both gene knockout and gene overexpression. The pLCRISPR/Cas9 system (Addgene) can be used to knockout single or multiple target genes. The guide RNA sequence (gRNA) can be designed using CRISPR direct online software (CR1SPRdirect). The highspecificity gRNA sequence for each target can be selected and cloned into plasmid pL CRISPR.SEFV.GFP (Lentiviral CRISPR-Cas9 delivery for SpCas9 and sgRNA, Addgene).

To verify the effectiveness of the gRNA, COS-1 cells can be transfected with the vector using Lipofectamin 2000 (Invitrogen), and the knockout efficiency can be verified by western blot analysis at 72 h post transfection.

pLentiN (lentiviral backbone for cloning and overexpression of target genes labeled with flag tag) vector can be used to overexpress target genes. The cDNA of the target gene can be inserted into the vector. The expression of the target protein can be confirmed by transient transfection of COS-1 cells with the vector followed by western blot analysis.

To package the viral particles, the confirmed vector pL-CRISPR.SFFV.GEP with inserted sgRNA or pLentiN with inserted cDNA can be co-transfected with pCMV-VSV-G (Envelope protein for producing Lentiviral particles) and pCMV-dR8.2 dvpr (packing plasmid for producing Lentiviral particles) into the HEK293T cell line at a ratio of 1:3:4. Two days after transfection, the media containing lentiviral particles from the transfected cells can be harvested and stored at 4° C. Corresponding empty vector can also be used to pack the viral particle, which can be used for control experiments.

Construction of stable cell lines:

The following stable cell lines are constructed using Crispr/Cas9 genome editing, mammalian gene expression, and lentivirus system from Addgene. (Note: Cell9 denotes gene knockout; Cell′ denotes gene overexpression; cNOS denotes double knockout or overexpression both nNOS and eNOS).

HaCaT cell lines: HaCaT^{nNOS-/-}, HaCaT^{eNOS-/-}, HaCaT^{cNOS-/-}, HaCaT^{nNOS}, HaCaT^{eNOS}, HaCaT^{cNOS}; HaCaT^{IKK-/-}, HaCaT^{IKK}, HaCaT^{p65-/-} (UVB only activates p65 subunit of NF-κ.B)

HEK293T cell lines: HEK29^{nNOS}, HEK293T^{eNOS}, and HEK293T^{cNOS}

SCC-13 cell lines: SCC-13^{nNOS-/-}, SCC-13^{eNOS-/-}, SCC-13^{cNOS-/-}, SCC-13^{nNOS}, SCC-13^{eNOS}, SCC-13^{cNOS}, SCC-13^{IKK-/-}, SCC-13^{IKK}.

A431 cell lines: A431^{nNOS-/-}, A431^{eNOS-/-}, and A431^{cNOS-/-}; A431^{nNOS}, A43e^{NOS-/-}, and A431^{cNOS./} A431IKK^{-/-}, A431^{IKK}.

Cell lines with target-gene knockout or overexpression can generated by infecting cells with pL CRISPR-target-gene or pLentiN-target-gene viral particles respectively. Briefly, the lentiviral particle containing media are directly added to cell culture media containing 8 μg/mL of polybrene. To establish stable cell lines, the fresh media with 1-3 μg/mL of puromycin can be placed on infected cells at 24 h post infection.

### EXAMPLES

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

### Example 1

cNOS in Regulation of NF-κB Activation, IKKα Expression, and Autophagy Post-UVB.

Described herein is a cNOS-centered NF-κB-IKKα signaling circuit in regulation of UVB-induced autophagy. FIG. 2 provides a model for UVB induced cNOS-NF-κB-IKKα regulated autophagy. UVB-induced NF-κB activation alone, or both pLCRISPR-eNOS and 24 h post-infection, the cells can be and then can be sham or UVB determined at 6 h after UVB treatment.

1.1.

Determination of the necessity and sufficiency of nNOS and eNOS in regulation of activation of NF-κB after UVB. cNOS has two isoforms (eNOS and nNOS) and has multiple functions.

a. To determine each cNOS for UVB-induced NF-κB activation: treat HaCaT, HaCaT^{nNOS-/-}, HaCaT^{eNOS-/-} and HaCaT^{cNOS-/-} with UVB, and determine NF-κB activation in the cells at 6 h post-irradiation).

b. To determine the sufficiency of each cNOS expression for restoration of the inducibility of NF-κB activity in cNOS-null HEK293T cells: treat HEK293T, HEK293T^{nNOS}, HEK293^{eNOS} HEK293T^{cNOS} cells with UVB, and determine NF-κB activation, including NF-κB DNA binding, translocation and phosphorylation at 6 h post-irradiation.

c. To confirm that cNOS-dependent NF-κB activation upon UVB irradiation is not cell line specific: repeat 1.1a with HEKn primary human keratinocytes. The cells are infected with pLCRISPR viral particle pLCRISPR-nNOS viral particles. At 24 h post-infection, the cells are selected with puromycin for one week, and then sham or UVB irradiated. NF-κB activation is determined at 6 h after UVB treatment.

d. To determine the effect of UVB doses on cNOS-mediated NF-κB activation: analyze NF-κB activation in HaCaT and HaCaT^{cNOS-/-} cells after treating with 5 mJ/cm² (dose for cell transformation assay), 50 mJ/cm² (˜50% MED) and 180 mJ/cm² (dose for mouse model).

e. To determine the effect of UVB doses on cNOS-mediated NF-κB activation. analyze NF-κB activation in HaCaT and HaCaT^{cNOS-/-} cells after treating with 5 mJ/cm² every other day for 10 days.

1.2.

Determination of the mechanism for maintaining IKKα protein levels when IKKα mRNA is reduced by UVB radiation.

UVB irradiation alone significantly reduces IKKα mRNA level, but not IKKα protein level. However, while cNOS or NF-κB inhibitors do not by themselves effect IKKα mRNA or protein level, all of them can lead to a reduction of the IKKα protein level, but not mRNA level after UVB irradiation.

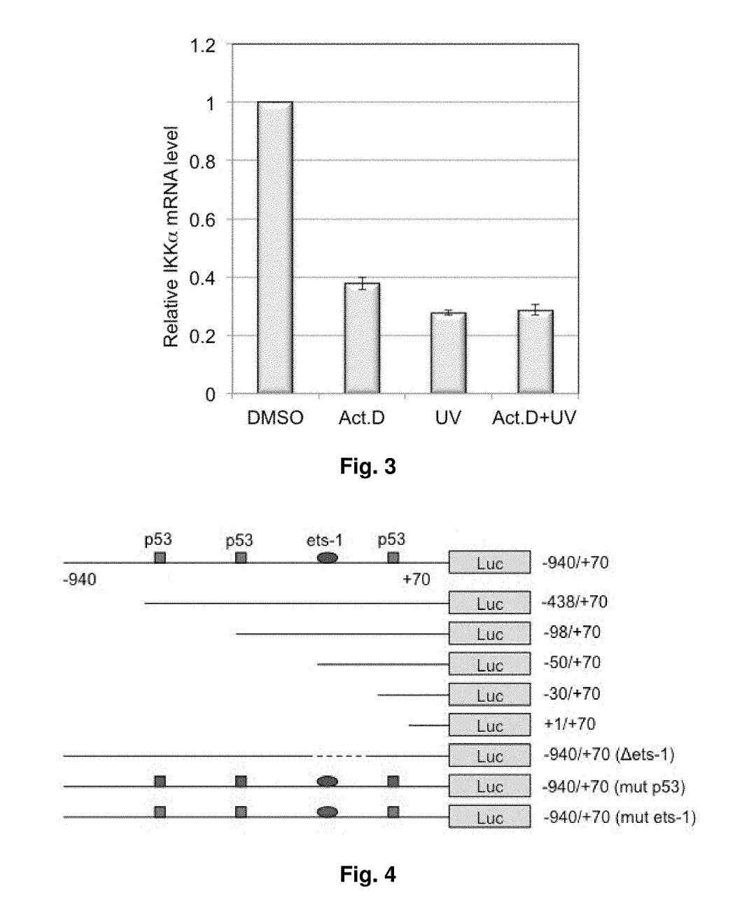

The data herein show that transcription inhibitor actinomycin D does not have a statistically significant effect on IKKα mRNA level post-UVB, showing that UVB inhibits IKKα mRNA transcription but not mRNA stability (FIG. 3).

The molecular mechanism of UVB-induced down-regulation of IKKα mRNA expression is determined (along with the involvement of the two mechanisms, increased protein stability and/or increased translation efficiency) for maintenance of constant IKKα protein level post-UVB. Also, the mechanism for NF-κB-mediated stabilization of IKKα post-UVB irradiation is determined.

a. To identify factor(s) that are involved in suppressing the IKKα promoter after UVB irradiation: use the Dual-Glo luciferase assay kit (E2920, Promega) to analyze the responses of full length and deletion-mutated promoters to UVB irradiation. Briefly, the full-length (−940/+70) promoter and a series of deletion mutants (−438/+70, −98/+70, −50/+70, 30/+70, +1/+70, −940/+70 del ets-1, −940/+70 mut p53, −940/+70 mut ets-1) of the IKKα promoter (FIG. 4) are constructed into a promoterless luciferase expression vector pGL3-basic (Promega). The deletion mutants were designed based since the IKKα promoter is negatively regulated by p53 and est-1. HaCaT cells are transiently co-transfected with the pGL3-IKKα-Luc vectors (empty, full or mutated IKKα promoter) and a Renilla luciferase expression vector. At 24 h post-transfection, the cells are UVB irradiated and the luciferase (firefly and Renilla) activities are determined from 0-6 h at 2 h intervals

The factor(s) that regulate IKKα transcription are identifiable using in silico analysis and protein-DNA binding EMSA. The roles of the identified factors in regulation of IKKα transcription after UVB irradiation are further confirmed using CRISPR/Cas9 knockout and EMSA.

b. To determine the mechanism of cNOS-mediated NF-κB activation in maintenance of IKKα protein levels after UVB irradiation: use ³⁵S-MeVCys metabolic pulse labeling and pulse-chase labeling to study translation efficiency and stability respectively. Briefly, HaCaT, HaCaT^{cNOS-/-} and HaCaT^{p65-/-} cells are sham or UVB irradiated. The cells are then pulse-labeled with ³⁵S-Met/Cys (100 μCi/mL) for 30 mm from 0 to 6 h after UVB irradiation at 1 h intervals. Cells are pulsechase labeled in parallel with ³⁵S-Met/Cys (100 μCi/mL) for 30 mm followed by 30 mm incubation in complete media. Total cell lysates are prepared after pulse- or pulse-chase labeling; and IKK protein are immunoprecipitated and analyzed on SDS-PAGE followed by autoradiography.

c. To elucidate the mechanisms of cNOS-mediated NF-κB activation in maintaining IKKα levels: determine whether the activated NF-κB stabilizes IKKα by forming a complex and/or promoting nuclear localization of IKKα. Briefly, HaCaT and HaCaT^{cNOS-/-} are sham or UVB irradiation. At 6 h post-UVB, the cytoplasmic and nuclear fractions are isolated. The p65 of NF-κB or IKKα in each fraction is immunoprecipitated and co-precipitated NF-κB(p65) or IKKα is determined by western blot analysis. The co-localization of NF-κB(p65) and IKKα are determined by co-immunostaining.

1.3.

Determination of the mechanism of cNOS-mediated NF-κB activation in regulation of apoptosis and autophagy after UVB irradiation.

The data indicate that LC3 did not aggregate in cNOS-null HEK293T cells after UVB irradiation (FIG. 1), showing cNOS may be required for UVB-induced autophagy. A cNOS-mediated NF-κB-IKKα signaling cascade is a controller for UVB-induced autophagy and cell death.

a. To determine the role of eNOS and/or nNOS in regulation of apoptosis and autophagy after UVB irradiation: sham and UVB irradiate HaCaT, HaCaT^{aNOS-/-}, HaCaT^{eNOS-/-} and HaCaT^{cNOS-/-} cells. At 6 and 24 h post-UVB, autophagy of the cells are determined by analysis of autophagy markers LC-I, LC3-II, and p62 by western blot. Autophagy can also be analyzed by transiently transfecting pcDNA3.1-GFPLC3B plasmid into the above cell lines and UVB irradiating the cells at 24 h post-transfection. The formation of autophagosomes in the cells is then detected at 6 h post-UVB using fluorescent dye-conjugated LC3 antibody and fluorescence microscopy as shown in FIG. 1. Apoptosis of the cells is determined by analysis of apoptosis protein markers by western blot and AnexinV/Pl staining by flow cytometry.

b. To determine whether cNOS-maintained IKKα expression is required for UVB-induced autophagy: treat HaCaT^{IKK-/-} cells with sham or UVB. Autophagy and apoptosis are determined at 6 and 24 h post UVB exposure as described in 1.3a. To show that IKKα plays a role in regulation of autophagy after UVB, repeat the experiment using HEKn with the pLCRISPR-IKKα viral particles and one-week puromycin selection as described in 1.1c.

c. To determine whether an increase of IKKα levels is sufficient to upregulate autophagy in UVB irradiated cells, HaCaT, HaCaT^{nNos-/-}, HaCaT^{eNOS-/-}, and HaCaT^{cNOS-/-} cells are infected with LentiN-IKKα viral particles and the cells are then cultured for a week to recover. The IKKα overexpression in the cells are the confirmed by western blot analysis. The IKKα overexpression cells are then treated with sham or UVB. Autophagy and apoptosis are determined at 6 and 24 h post UVB treatment as described in 1.3a.

Results of Example 1:

Expressing either nNOS or eNOS can be necessary and sufficient for induction of NF-κB activation and maintenance of IKKα expression levels in the early phase of UVB irradiation.

Double knockout or knockout of nNOS or eNOS individually can impair the inducibility of NF-κB and reduce IKKα expression levels upon UVB irradiation.

Maintaining a relatively constant level of IKKα is needed for UVB induced autophagy.

Reducing the expression of IKKα can inhibit autophagy induced by UVB. However, overexpression of IKKα cannot induce cell autophagy, but can sensitize cell to UVB-induced autophagy. Identification of the IKKα mRNA translational addition shows the mechanisms that regulate IKKα transcription factor(s) that regulate the IKKα promoter; i.e., by determining efficiency, and by identifying the pathway that regulates IKKα protein stability after UVB treatment.

### Example 2

Assessment of the Role(s) of cNOS in Regulation of UVB-Induced DNA Damage, Cell Death, and Skin Cancer Formation and Progression.

The data indicate that expression of cNOS is required for UVB-induced cell autophagy (FIG. 1).

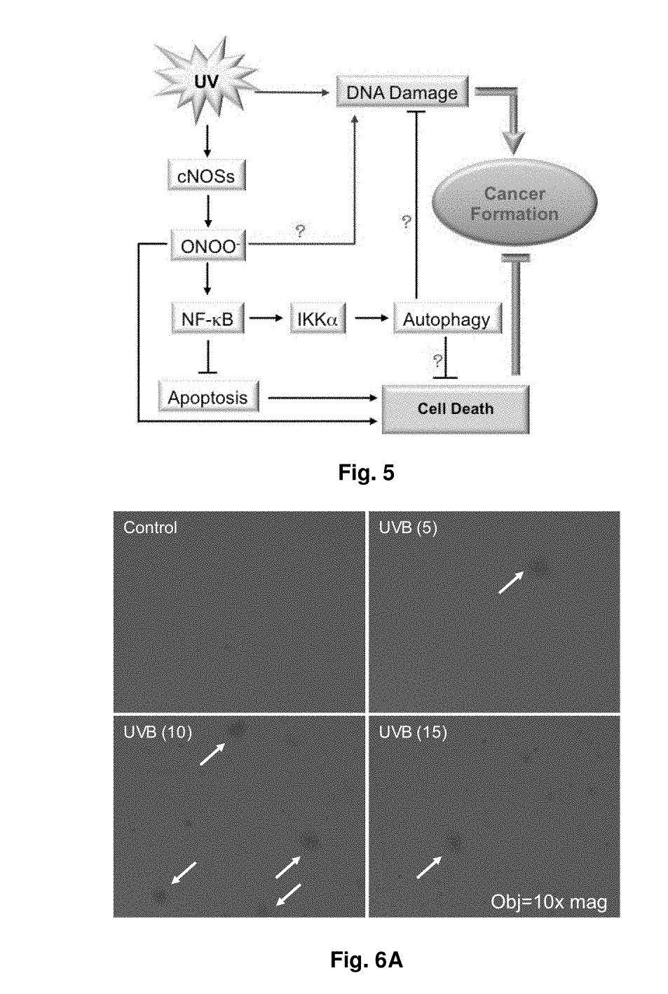

Also, cNOS plays a critical role in regulation of skin carcinogenesis by promoting a simultaneous increase in both ONOO⁻-induced DNA damage and IKKα mediated autophagy after UVB irradiation (FIG. 5).

The contribution of cNOS to UVB-induced DNA damage and IKKα-mediated autophagy, as well as their roles in regulation of keratinocyte transformation and squamous cell carcinoma (SCC) progression post-UVB, have been determined. The data show that cNOS is involved in the regulation of skin cell transformation and skin cancer progression.

2.1.

Determination of the role of cNOS and cNOS-mediated autophagy in regulation of UVB-induced DNA damage and cell death. cNOS and cNOS-NF-κB-IKKα cascade contribute to in UVB-induced DNA damage and cell death.

a. To determine the contribution of each cNOS to DNA damage in UVB-irradiated cell: treat HaCaT, HaCaT^{nNOS-/-}, HaCaT^{eNOS-/-}, and HaCAT^{cNOS-/-} cells with sham and UVB irradiation; then determine the total amount of and kinetics of removing cyclobutane pyrimidine dimers (CPD), (6-4)photoproducts (6-4PP) and 8-oxo-2′-deoxyguanosine (8-oxodG) at 0, 6, 24 and 48 h post-UVB by slot blot analysis using corresponding antibodies. The intensity of the bands are semi quantitated using ImageJ (Version 1.42k, NIH) .

b. To determine the role of cNOS-NF-κB-IKKα-mediated autophagy in regulation of UVB-induced DNA-damage repair: pre-treat HaCaT, HaCaT^{nNOS-/-}, HaCaT^{eNOS-/-}, and HaCAT^{cNOS-/-} cells with vehicle or an autophagy inhibitor Spautinl (10 nM) for 6 h; then, treat with sham and UVS irradiation. The cells are cultured in the same medium with or without Spautinl until harvest. Again, the total amount of and kinetics of removing CPD, 6-4PP and 8-oxodG at 0, 6, 24 and 48 h post UVB are determined and semi-quantitated as described above in 2.1a.

c. To determine whether cNOS mediated autophagy plays a major role in regulation of UVB-induced cell death: pre-treat HaCaT, HaCaT^{nNOS-/-}, HaCaT^{eNOS-/-}, and HaCAT^{cNOS-/-} cells with vehicle or an autophagy inhibitor Spautin1 (10 nM) for 6 h; then, treat with sham and UVB irradiation. The cells are continuously cultured in the medium with or without Spautinl until harvest at 6 h and 24 h post-U VS irradiation. Autophagy, apoptosis, cell viability and recovery are determined as described herein.

2.2.

Determination of the role of cNOS-IKKα cascade in regulation of UVB-induced transformation of keratinocytes.

cNOS-IKKα cascade plays a role in regulation of UVB-induced skin cell transformation.

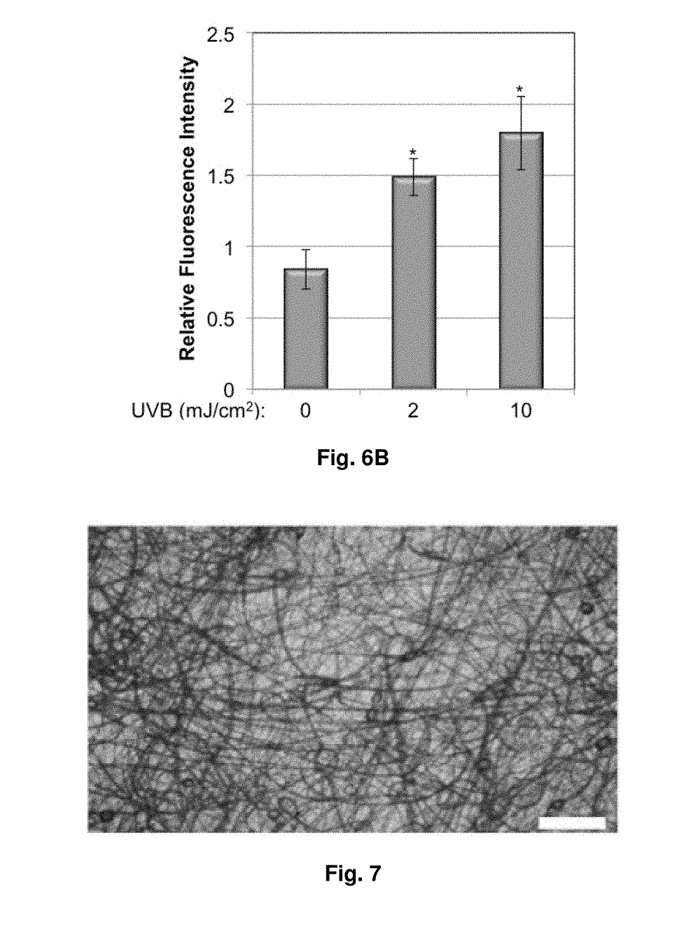

a. To determine the chemopreventive effect of cNOS inhibitor for UVB-induced cell transformation: treat HaCaT cells with sham or drug (L-NAME) (1 mM) from 1 h before to 6 h after UVB (10 mJ/cm²) treatment every other day for 10 days. Cells with the same drug treatment and sham UVB irradiation are used as control. After the drug and UVB treatment, cell transformation is determined using a soft-agar assay as shown in FIG. 6, and western blot analysis of oncogenic biomarkers such as keratin 5 and MMP9. The role of cNOS in regulation of UVB-induced cell transformation is confirmed by performing the cell transformation experiment using the NOS knockout cells including HaCaT^{nNOS-/-}, HaCaT^{eNOS-/-} and HaCaT^{cNOS-/-}.

b. To determine whether an increased expression of each cNOS can be correlated with an increased numbers of transformed cells after UVB irradiation: treat the stable NOS overexpression cells, including HaCaT^{nNOS}, HaCaT^{eNOS} and HaCaT^{cNOS} with sham or UVB before the cell transformation analyses, as described in 2.2a.

c. To determine whether IKKα expression level plays a role in UVB-induced cell transformation: treat the stable IKKα knockout HaCaT^{IKK-/-} and IKKα overexpression HaCaT^{IKK} cells: with sham or UVB before the cell transformation analyses is performed as described in 2.2a.

2.3.

The role of cNOS-maintained IKKα expression in regulation of invasiveness and EMT of SCC cells post-UVB.

Nitric oxide-releasing nonsteroidal anti-inflammatory drugs (NONO-NSAIDs) reduce adhesive affinity of melanoma cells to endothelial and extracellular matrix (ECM) proteins, as well as reduce the expression of epithelial mesenchymal transition (EMT) markers in UVB-induced skin cancers. Autophagy-regulated p62 plays a role in regulation of SCC cell proliferation and migration. The data herein show that cNOS expression is needed for UVB-induced autophagy (FIG. 1). That is, cNOS and IKKα play roles in regulation of migratory behaviors and EMT of SCC cells.

a. To determine the necessity of early activation of cNOS in regulation of SCC progression after UVB irradiation: treat the cells with sham or drug (L-NAME) (1 mM) from 1 h before to 6 h after UVB irradiation. The invasiveness of the cells is determined at 6 and 12 h after UVB irradiation using a transwell migration assay. EMT of the cells is determined at 6 and 12 h after UVB irradiation by analysis of the expression of EMT markers (vimentin and E-cadherin etc.) using immunofluorescent staining and western blot analysis.

b. To determine whether cNOS expression levels are correlated with SCC progression: determine the extent of the effect of NOS overexpression and knockout on invasiveness and EMT of human epidermal SCC lines SCC-13 and A431. Briefly, NOS overexpression cells, including SCC-13^{nNOS}, SCC-13^{eNOS}, SCC-13^{nNOS}, A431^{nNOS}, A431^{eNOS}, and A431^{cNOS}; and NOS knockout cells including SCC-13^{nNOS-/-}, SCC-13^{eNOS-/-}, SCC-13^{cNOS-/-}, A431^{nNOS-/-}, A431^{eNOS-/-}, and A431^{cNOS-/-}, are treated with sham or UVB before the analysis of cell invasiveness and EMT as described in 2.3a.

c. To determine the requirements for IKKα activity for SCC progression: determine the extent of effect of IKKα overexpression and knockout on invasiveness and EMT of human epidermal SCC lines SCC-13 and A431. Briefly, IKKα overexpression cells, including SCC-13^{IKK} and A431^{IKK} and IKKα knockout cells including SCC13^{IKK-/-} and A431^{IKK-/-}, are treated with sham or UVB before the analysis of cell invasiveness and EMT as described in 2.3a.

cNOS knockout can reduce DNA damage and increase cell viability. cNOS activation is needed for UVB-induced autophagy; thus, treating cells with a cNOS inhibitor can reduce UVB-induced HaCaT transformation. The autophagy inhibitor Spautinl can inhibit UVB-induced autophagy and reduce cell viability of wild-type HaCaT cells but have less effect on HaCaT cells with cNOS, p65 and IKKα knockout. The cNOS inhibitor and cNOS overexpression can reduce invasiveness and the EMT of SCC cells due to a mechanism similar to that discussed above. The SCC cells that are co-cultured with HUVEC cells have reduced invasiveness and EMT after UVB irradiation. IKKα knockout can have similar effects on HaCaT cell transformation as well as invasiveness and EMT of SCC cells. Overexpression of IKKα can increase HaCaT cell transformation and invasiveness and EMT of SCC cells due to the fact that IKK can not only promote autophagy but can also activate NF-κB, which promotes cell survival and cancer progression. However, this does not mean that overexpression of cNOS can increase HaCaT transformation after UVB irradiation. In fact, it is now believed that the overexpression of each cNOS can also reduce cell transformation because a higher level of NO production can shift NO/ONOO⁻ towards a healthier balance, and thus might reduce ER stress and autophagy.

### Example 3

Determination of the Roles of cNOS in UVB-Induced Photocarcinogenic Responses, Non-Melanoma Cancer Formation, and Tumor Progression in 3D Co-Culture and Mouse Models.

Inhibition of cNOS can reduce the incidence of UVB-induced non-melanoma skin cancer formation, as well as suppress tumor progression. cNOS produces NO and O₂⁻, which quickly react with each other to form ONNO⁻ post-UVB. Since both NO and ONOO⁻ can diffuse to adjacent cells through membrane permeation or gap junctions, the production of both these molecules in adjacent cells can have an effect on target cells. The effect of cocultured eNOS-rich endothelial cells on UVB-induced keratinocyte transformation and SCC progression using a customized 3D cell co-culture model is shown in FIG. 7.

The effect of cNOS knockout and topical treatment of a broad cNOS inhibitor L-NAME on UVB-induced skin cancer formation and progression using SKH-1 hairless mouse models.

3.1.

Customized 3D cell co-culture system for analysis of the importance of adjacent eNOS-rich endothelial cells on UVB-induced skin cell transformation and SCC progression.

Cells grown in 2D culture lack cell-cell communication and ECM supporting structure, which often makes them differ in response to stimuli compared to cells in tissues, organs and animal models. To overcome this drawback, 3D cell culture systems have been developed. However, commercial 3D culture systems lack flexibility and are not optimized for all cell lines. Thus, described herein is a different 3D cell culture system, which can be optimized. The nanofiber scaffold (FIG. 7) for 3D cell culture is prepared by electrospinning of polymer solutions, which is a technique to produce nano- to micro-scaled polymer fibers to build the micro-structure inside the scaffold. The solution of bio-friendly polymer material is injected into a high-voltage electronic field. The strong electronic field is able to stretch thin polymer fibers, which are collected and automatically form suitable scaffolds. This system is very convenient to modify. The type of polymer, the solvent, the voltage, and the solution injection rate can easily be modified; and all have an impact on the characteristics of the fibers.

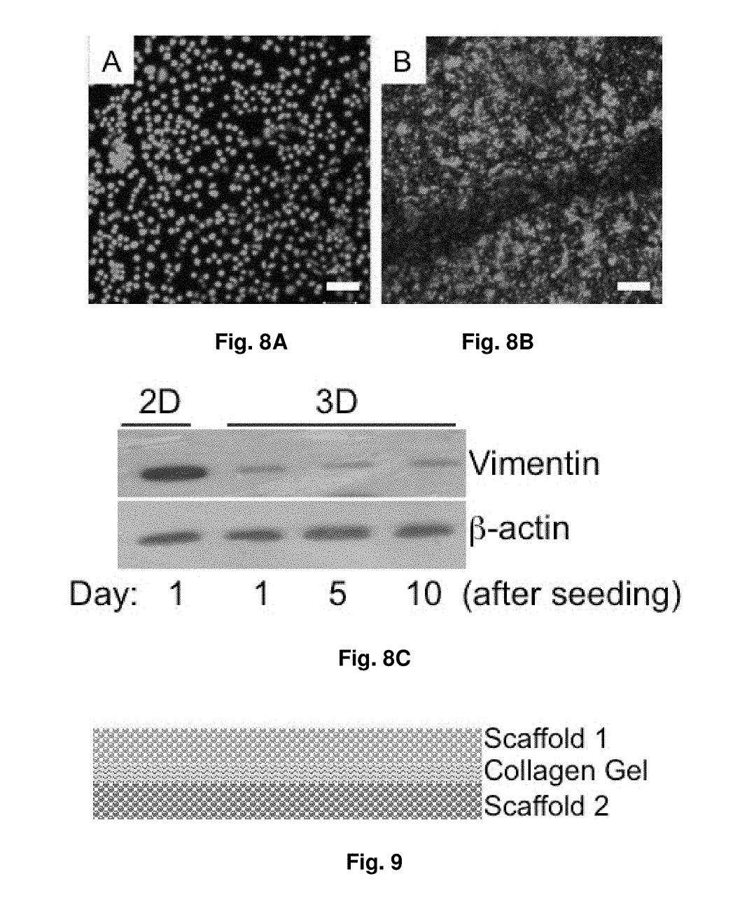

The data demonstrate that H 1299 human non-small cell lung carcinoma cells cultured on regular culture plates or the 3D system (FIG. 8A-FIG. 8B) express different levels of vimentin, an EMT marker (FIG. 8C), which can affect their migration behavior and invasiveness.

The two-layer 3D cell co-culture system (FIG. 9) is used to analyze the effect of adjacent eNOS-rich endothelial cells on the response of target keratinocytes and SCC cells to UVB irradiation.

a. Determination of the effect of adjacent endothelial cells on UVB induced transformation of keratinocytes, HaCaT and HUVEC cells are cultured separately on the 3D scaffold, and then the cells are stacked as HaCaT/HaCaT or HaCaT/HUVEC with a collagen gel spacer between the layers (FIG. 9). The cells are treated with sham and UVB (10 mJ/cm²) every other day for 10 days. The two layers are then separated and HaCaT cell transformation is determined as described in 2.2a.

b. To determine the effect of adjacent endothelial cells on SCC progression post-UVB, SCC-13, A431 and HUVEC cells are cultured separately on the 3D scaffold, and then the cells are stacked as SCC-13/SCC-13, SCC-13/HUVEC, A431/A431 orA431/HUVEC with a collagen gel spacer between the layers. The cells are treated with sham and UVB irradiation. At 6 and 12 h after UVB irradiation the two scaffolds are separated, and the invasiveness and EMT of SCC cells is analyzed as described in 2.3a.

3.2.

Determination of the role of cNOS in regulation of UVB-induced photocarcinogenic responses in mice.

Both apotosis and autophagy regulate UVB-induced skin carcinogenesis. cNOS activation is critical for maintaining IKKα levels and inducing autophagy in UVB-irradiated cells. cNOS has a role in regulation of NP-κ.B activation, IKKα expression, apoptosis, autophagy, and short-term photocarcinogenesis.

a. To determine the role of nNOS and/or eNOS in regulation of UVB-induced NF-κB activation, IKKα expression and autophagy, mice including SKH-1 (WT), SKH-1 (nNOS^{-/-}), SKH-1 (eNOS^{-/-}) and SKH-1 (cNOS^{-/-}), are UVB (180 mJ/cm²) irradiated, and skin sections are prepared at 12 h after UVB irradiation for analysis. NF-κB localization and IKKα levels are determined by immunohistochemical staining. The expression of autophagy markers (LC3-I, LC3-II and p62) in total proteins extracted from skin is determined using western blot analysis. The level of p62 in the skin tissue is also determined using immunohistochemical staining.

b. To determine the potential chemopreventive effect of a cNOS inhibitor, SKH-1 (WT) is topically treated with L-NAME (2 mM) 1 h before sham or UVB (180 mJ/cm²) treatment and skin section are prepared at 12 h after UVB irradiation. Short-term photocarcinogenesis is then analyzed as follows: (I) Skin edema is studied by evaluating the increases in hi-fold skin thickness using a micrometer. The increase in bifold skin thickness is obtained by subtracting values for the control animals. (II) Epidermal hyperplasia is studied by histopathological examination. Paraffin-embedded skin tissue is H&E stained and examined microscopically for hyperplasia. Epidermal hyperplasia is determined by assessing vertical epidermal thickness and number of vertical epidermal layers. (III) Epidermal ornithine decarboxylase (ODC) induction is studied by immunohistochemical staining.

3.3

The role of cNOS in UVB-induced non-melanoma cancer formation in mice.

a. To determine the role of nNOS and/or eNOS in UVB-induced skin cancer formation, mice, including SKH-1 (WT), SKH-1 (nNOS^{-/-}), SKH-1 (eNOS^{-/-}) and SKH-1 (cNOS^{-/-}), are sham or UVB (180 mJ/cm) irradiated twice a week for 28 weeks. Skin and tumor sections are then prepared and characterized. Briefly, tumor incidence are visually counted. The tumor size is measured by determining two perpendicular dimensions with calipers, and the volume is calculated using the formula (a×b²)/2, (a=longer dimension, b=smaller dimension). Tumor characteristics are evaluated by histopathological analysis. The sections (4 μm) of formalin-fixed and paraffin-embedded skin tissue are mounted on slides, and stained with hematoxylin and eosin. Tumor characteristics are examined under the microscope.

b. To determine the chemoprevention effect of a cNOS inhibitor on UVB-induced skin cancer formation, SKH-1 (WT) are topically treated with L-NAME (2 mM) 1 h before sham or UVB (180 mJ/cm²) irradiation. The treatment is repeated twice a week for 28 weeks. Skin and tumor sections are then prepared and characterized during the period of 20-28 week with 4 weeks intervals as described in 3.3a.

### Example 4

Using L-NAME for Chemoprevention of UVB-Induced Skin Carcinogenesis

4.1

L-NAME protects DNA in keratinocytes from UVB-induced damage.

To assess the effect of cNOS inhibitor on UVB-induced DNA damage in keratinocutes, HaCaT cells were treated with DMSO (solvent) or L-NAME (1 mM) for 1 hour prior to UVB radiation (10 mJ/cm²). At 30 mM post-UVB, the cells were collected and DNA damage was determined using a neutral comet assay kit following manufacture's protocol (Trevigen, Gaithersburg, Md.).

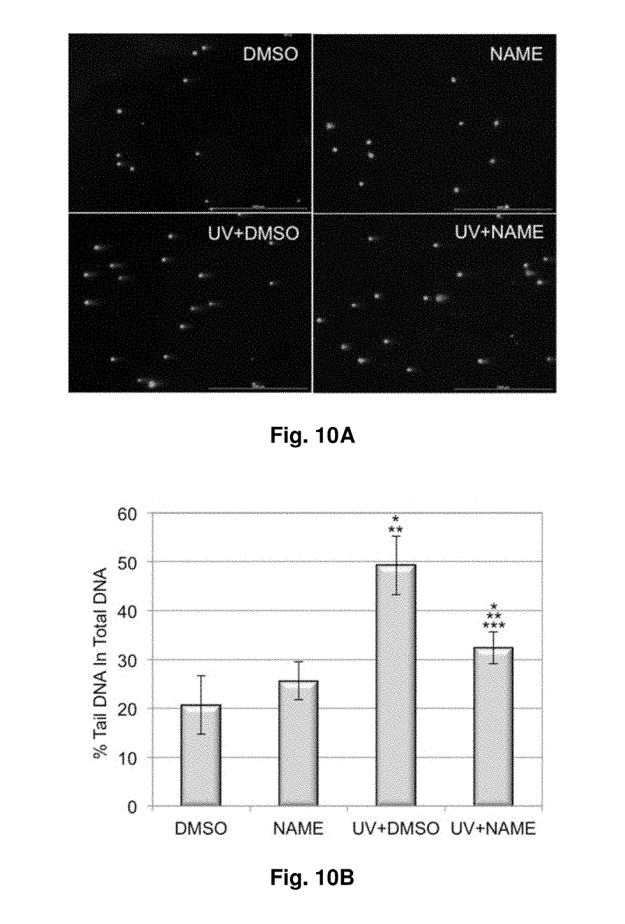

The treatment of L-NAME reduced the percentage of UVB-induced tail DNA content from 49.2% to 32.4% (FIGS. 10A-10B). Considering the 20.7% or 25.6% tail DNA content in DMSO or L-NAME alone, the treatment of L-NAME reduced the UVB-induced DNA breakage in keratinocytes by 58.9% or 71.2% respectively. The % of DNA damage reduction was calculated as (the Control can be DMSO or L-NAME alone):

$$\frac{\begin{matrix}( \% tail {DNA}_{UVB + DMSO} - \% tail {DNA}_{DMSO} ) - \\ ( \% tail {DNA}_{UVB + NAME} - \% tail {DNA}_{Control} )\end{matrix}}{\% tail {DNA}_{UVB + DMSO} - \% tail {DNA}_{DMSO}}$$

4.2

L-NAME protects keratinocyte from UVB-induced transformation.

To determine the chemoprevetive function of cNOS inhibitor for UVB-induced skin cell transformation, cells were treated with sham or UVB every other day for 14 days in the presence or absence of L-NAME (1 mM in media) all time. The cells were detached from plate and same amount of cells were seeded in soft-agar in 96-well microtiter plate. At day 10, the cells alone with soft-agar were solubilized in the wells using Cytoselect buffer (Cell Biolabs, Inc., San Diego, Calif.) and the amount of transformed cells was determined by fluorescence intensity using a Spectra Max M2 fluorescent platereader (Molecular Devices).

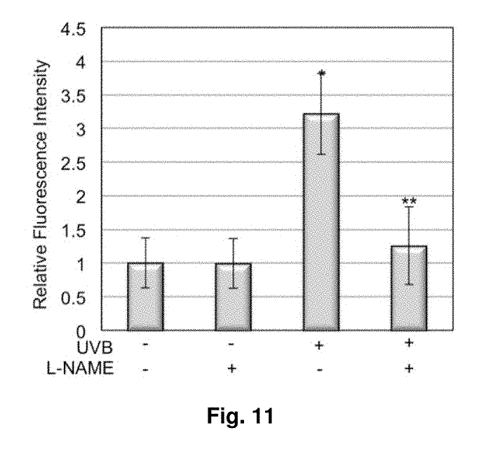

The data show that the treatment of L-NAME reduced the reading of transformed cells from 3.2 to 1.3 (FIG. 11). These results indicate that the presence of L-NAME almost totally suppresses UVB-induced transformation of keratinocytes.

### Example 5

Pharmaceutical Compositions

A pharmaceutical composition as described herein may be formulated with any pharmaceutically acceptable excipients, diluents, or carriers. A composition disclosed herein may comprise different types of carriers depending on whether it is to be administered in solid, liquid, or aerosol form, and whether it needs to be sterile for such routes of administration as injection. Compositions disclosed herein can be administered in a suitable manner, including, but not limited to topically (i.e., transdermal), subcutaneously, by localized perfusion bathing target cells directly, via a lavage, in creams, in lipid compositions (e.g., liposomes), formulated as elixirs or solutions for convenient topical administration, formulated as sustained release dosage forms, or by other method or any combination of the forgoing as would be known to one of ordinary skill in the art (see, for example, Remington's Pharmaceutical Sciences, 2003, incorporated herein by reference).

The compositions provided herein are useful for treating animals, such as humans. A method of treating a human patient according to the present disclosure includes the administration of a composition, as described herein.

The phrases “pharmaceutical” or “pharmacologically acceptable” refer to molecular entities and compositions that produce no adverse, allergic, or other untoward reaction when administered to an animal, such as, for example, a human. A carrier or diluent may be a solid, semi-solid, or liquid material which serves as a vehicle, excipient, or medium for the active therapeutic substance. Some examples of the diluents or carriers which may be employed in the pharmaceutical compositions of the present disclosure are lactose, dextrose, sucrose, sorbitol, mannitol, propylene glycol, liquid paraffin, white soft paraffin, kaolin, fumed silicon dioxide, microcrystalline cellulose, calcium silicate, silica, polyvinylpyrrolidone, cetostearyl alcohol, starch, modified starches, gum acacia, calcium phosphate, cocoa butter, ethoxylated esters, oil of theobroma, arachis oil, alginates, tragacanth, gelatin, syrup, methyl cellulose, polyoxyethylene sorbitan monolaurate, ethyl lactate, methyl and propyl hydroxybenzoate, sorbitan trioleate, sorbitan sesquioleate and oleyl alcohol, and propellants such as trichloromonofluoromethane, dichlorodifluoromethane, and dichlorotetrafluoroethane.

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols, and mixtures thereof, and in oils. Under ordinary conditions of storage and use, these preparations contain a preservative to prevent the growth of microorganisms. The pharmaceutical forms suitable for use include sterile aqueous solutions or dispersions and sterile powders for the extemporaneous preparation of sterile solutions or dispersions. In certain cases the form should be sterile and should be fluid to the extent that easy injectability exists. It should be stable under the conditions of manufacture and storage and may optionally be preserved against the contaminating action of microorganisms, such as bacteria and fungi. The carrier can be a solvent or dispersion medium containing, for example, water, ethanol, a polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, such as, but not limited to, sugars or sodium chloride. Prolonged absorption of the injectable compositions can be brought about by the use in the compositions of agents delaying absorption such as, for example, aluminum monostearate, or gelatin.

Some variation in dosage can necessarily occur depending on the condition of the subject being treated. The person responsible for administration can, in any event, determine the appropriate dose for the individual subject.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream, and petrolatum as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

It is further envisioned the compositions disclosed herein may be delivered via an aerosol. The term aerosol refers to a colloidal system of finely divided solid or liquid particles dispersed in a liquefied or pressurized gas propellant. The typical aerosol comprises of a suspension of active ingredients in liquid propellant or a mixture of liquid propellant and a suitable solvent. Suitable propellants include hydrocarbons and hydrocarbon ethers. Suitable containers can vary according to the pressure requirements of the propellant. Administration of the aerosol can vary according to subject's age, weight, and the severity and response of the symptoms.

Dosage

The actual dosage amount of a composition disclosed herein administered to an animal or human patient can be determined by physical and physiological factors such as body weight, severity of condition, the type of disease being treated, previous or concurrent therapeutic interventions, idiopathy of the patient, and the route of administration. Depending upon the dosage and the route of administration, the number of administrations of a preferred dosage and/or an effective amount may vary according to the response of the subject. The pharmaceutical compounds are generally effective over a wide dosage range. The practitioner responsible for administration can, in any event, determine the concentration of active ingredient(s) in a composition and appropriate dose(s) for the individual subject.

In certain embodiments, pharmaceutical compositions may comprise, for example, at least about 0.1% of an active compound. In other emboxdiments, an active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage can be obtained in any given unit dose of the compound. Factors such as solubility, bioavailability, biological half-life, route of administration, product shelf life, as well as other pharmacological considerations can be contemplated by those preparing such pharmaceutical formulations, and as such, a variety of dosages and treatment regimens may be desirable.

In some embodiments, the compositions further include one or more additional active ingredients. The preparation of a pharmaceutical composition that contains at least one compound or additional active ingredient can be known to those of skill in the art in light of the present disclosure, as exemplified by Remington's Pharmaceutical Sciences, 2003, incorporated herein by reference. Moreover, for animal (e.g., human) administration, it can be understood that preparations should meet sterility, pyrogenicity, and general safety and purity standards as required by the FDA Office of Biological Standards.

Cosmetic Compositions

In another other exemplary embodiment, the compositions described herein can be formulated as non-therapeutic, and in particular, a cosmetic product.

“Cosmetic composition” means compositions suitable for topical application on mammalian keratinous tissue.

“Keratinous tissue” refers to keratin-containing layers disposed as the outermost protective covering of mammals which include, but are not limited to, skin, hair, nails, and cuticles.

“Effective amount” means an amount sufficient to induce one or more biological effects as described herein.

Methods of Use

The cosmetic compositions disclosed herein may be applied to one or more skin surfaces and/or one or more mammalian keratinous tissue surfaces as part of a user's daily routine or regimen. Additionally or alternatively, the cosmetic compositions herein may be used on an “as needed” basis. In some examples, an effective amount of the cosmetic composition may be applied to the target portion of the keratinous tissue or skin. In some examples, the cosmetic composition may be provided in a package with written instructions detailing the application regimen.

The method may include a step of identifying a target portion of keratinous tissue or skin. The method may also include a step of identifying a skin surface for treatment with the cosmetic composition for improving skin condition. The skin surface may be identified by the user or a third party such as a dermatologist, cosmetician, or other individual or even by a combination of different individuals. Identification may be done, for example, by visual inspection of the skin surface in need of treatment based on size and/or color. Identification may also be done by either custom-made or commercially available imaging devices.

Skin surfaces may include those not typically covered by clothing such as facial skin surfaces, hand and arm skin surfaces, foot and leg skin surfaces, and neck and chest skin surfaces (e.g., decolletage). For example, areas identified for treatment may include areas such as the forehead, perioral, chin, periorbital, nose, and/or cheek skin surfaces. In another example, the cosmetic composition may be applied to any facial skin care surface and/or any other skin surface identified as in need of treatment by the cosmetic composition. In some examples, one or more of these skin surfaces may be identified as needing treatment and one or more of these skins surfaces may be treated with the cosmetic composition.

The method may comprise a step of applying the composition to the skin surface, which may or may not have been previously identified. Many regimens exist for the application of the cosmetic composition. The cosmetic composition may be applied as needed and/or at least once a day, twice a day, or on a more frequent daily basis, during a treatment period. Non-limiting examples of the treatment periods may be between about 1 week and about 12 weeks, between about 4 weeks and about 12 weeks, and/or between about 4 weeks and about 8 weeks. In another example, the treatment period may extend over multiple months (i.e., 3-12 months) or multiple years. In another example, the cosmetic composition may be applied least once a day during a treatment period of at least about 4 weeks or at least about 8 weeks. In another example, the cosmetic composition may be applied twice a day during a treatment period of at least about 4 weeks or 8 weeks. In another example, the cosmetic composition can also be applied to at least one skin surface area at least once per day, twice per day, or three times per day for a period of 7, 14, 21, or 28 days or more. When applied twice daily, the first and second applications may be separated by at least 1 to about 12 hours. The cosmetic composition may be also applied in the morning and/or in the evening before bed. The treatment period should be a sufficient time to provide an improvement in the skin surface but need not be so. For general application to keratinous tissue and, particularly a facial skin surface, the dosed amount of the cosmetic composition may be between about 1 to about 50 microliters/cm² per application (i.e., per single application to the skin surfaces).

Packaging of the Composition

After formulation, the composition is packaged in a manner suitable for delivery and use by an end user. In one embodiment, the composition is placed into an appropriate dispenser and shipped to the end user. Examples of final container may include a pump bottle, squeeze bottle, jar, tube, capsule or vial.

The compositions and methods described herein can be embodied as parts of a kit or kits. A non-limiting example of such a kit comprises the ingredients for preparing a composition, where the containers may or may not be present in a combined configuration. In certain embodiments, the kits further comprise a means for administering the composition, such as a topical applicator, or a syringe. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive, CD-ROM, or diskette. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

All publications, including patents and non-patent literature, referred to in this specification are expressly incorporated by reference herein. Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

1. A method of treating a skin cell of a subject, comprising:
administering a constitutive nitric oxide synthases (cNOS) inhibitor composition to the subject in an amount sufficient to inhibit DNA damage in the skin cells caused by exposure to UVB irradiation, and/or to protect the skin cells from UVB-induced transformation;
wherein the cNOS inhibitor composition comprises an endothelial NOS (eNOS) and/or a neuronal NOS (nNOS).

2. The method of claim 1, wherein the cNOS inhibitor composition comprises at least one of: L-N^{G}-nitro-arginine methyl ester (L-NAME) and NG-monomethyl-L-arginine (L-NMMA).

3. The method of claim 1, wherein the composition is in the form of a topical composition.

4. The method of claim 3, wherein the topical composition is in the form of a spray, mist, aerosol, lotion, cream, solution, oil, gel, ointment, paste, emulsion, suspension injectable and/or transdermal application.

5. The method of claim 1, wherein the skin of the subject is normal and healthy, and wherein the composition is applied to the skin for a time and in an amount sufficient to cause a reduction in UV damage to skin cells.

6. A composition comprising: an effective amount of a constitutive nitric oxide synthase (cNOS) inhibitor sufficient to inhibit DNA damage in the skin cells caused by exposure to UVB irradiation, and/or to protect the skin cells from UVB-induced transformation;
wherein the cNOS inhibitor comprises an endothelial NOS (eNOS) and/or a neuronal (nNOS); and,
one or more acceptable excipients, diluents and/or carriers and/or encapsulated.

7. The composition of claim 6, wherein the cNOS inhibitor comprises comprises at least one of: L-N^{G}-nitro-arginine methyl ester (L-NAME) and NG-monomethyl-L-arginine (L-NMMA).

8. The composition of claim 6, wherein the composition is in the form of a topical composition.

9. The composition of claim 8, wherein the topical composition is in the form of a spray, mist, aerosol, liquid, lotion, cream, solution, oil, gel, ointment, paste, emulsion, suspension, injectable and/or transdermal application.

10. The composition of claim 6, formulated as a therapeutic pharmaceutical composition.

11. The composition of claim 6, formulated as a cosmetic composition.

12. A method of chemoprevention of a pathological condition related to cell damage caused by UV irradiation, comprising: administering to an individual an effective amount of the composition of claim 10, wherein the composition is effective in ameliorating the effects of the pathological condition.
